(19) United States
(12) Patent Application Publication
(10) Pub. No.: US 2011/0160260 A1
(43) Pub. Date: Jun. 30, 2011

(54) Method of Treating a Neurodegenerative Disorder

(75) Inventor: Ying-Jui Ho, Tainan (TW)

(73) Assignees: Ying-Jui Ho, Taichung City (TW); Chung Shan Medical University, Taichung City (TW); T.L. & G.M. Chemical Company, Taichung City (TW)

(21) Appl. No.: 12/940,401

(22) Filed: Nov. 5, 2010

(30) Foreign Application Priority Data
Dec. 29, 2009 (TW) 098145509

(51) Int. Cl. A61K 31/42; A61P 25/28; A61P 25/18

(52) U.S. Cl. 514/380

(57) ABSTRACT

The present invention provides a method of treating a neurodegenerative disorder in a patient, comprising administration to the patient a therapeutically effective amount of D-cycloserine and its derivatives.

### FIELD OF THE INVENTION

This invention relates to a method of treating a neurodegenerative disorder, comprising administration of D-cycloserine or its derivatives.

### DESCRIPTION OF PRIOR ART

Previous studies have demonstrated that hyperactivation of glutamatergic system or over stimulation of N-methyl-D-aspartate (NMDA) receptors causes excitotoxicity in the nervous system and results in neurodegenerative disorders (Park, et al., Neurobiology of Aging, 2000, 21: 771-781). Therefore, it's expected that neurodegeneration and the dysfunction of mood, psychotic and cognitive derived from it could be improved by using the drug to modulate the activity of NMDA receptors.

1-methyl-4-phenyl-1,2,3,6-tetrahydropyridine (MPTP), a dopaminergic (DAergic) neurotoxin that selectively damages DAergic cells in the substantia nigra pars compacta (SNc), is widely used to induce animal models of Parkinson's disease in rodents and primates (Ferro et. al., Journal of Neuroscience Methods, 148: 78-87, 2005). When glutamate and glycine bind to the NMDA receptor, the calcium channel is able to be opened, and calcium ions influx to the cell. Treatment with MPTP causes not only DAergic degeneration but also over releasing of glutamate and thus led to excitotoxicity and neuronal cell death (Raju, et al., Eur J Neurosci, 2008, 27: 1647-58, Chassain et al., J Neurochem, 2008, 105: 874-82).

D-cycloserine (DCS), D-4-amino-3-isoxazolidone, is used as a broad-spectrum antibiotic. It is known that DCS, a partial agonist of NMDA receptor, is able to bind to glycine binding site on the NMDA receptor. Co-administration of DCS and typical antipsychotics has been reported to improve negative syndromes in patients with schizophrenia (Duncan, et al., Schizophrenia Research, 2004, 71: 239-248)

U.S. Pat. No. 6,551,993 disclosed that DCS improves inattention syndrome in Parkinson's disease and schizophrenia. However, there was no report on the effects of DCS on neuronal inflammation, neurodegeneration, and cognitive deficits resulted from the death of DAergic neurons. In addition, U.S. Pat. No. 7,160,913B2 disclosed that co-administration of DCS with levodopa and benserazide effectively increased the therapeutic effect of levodopa and decreased the side effect caused by levodopa, for example dystonia and dyskinesia. Although the embodiment 1 and FIG. 1 in the patent asserted that DCS facilitated the effects of levodopa and benserazide, the result reported in that patent was caused by the treatment of ACPC rather than DCS. Furthermore, FIG. 1 in the patent demonstrated the effect of combination of ACPC with levodopa was lower than that of administered levodopa alone on the contrary. Therefore, it is hard to presume that the inventor had provided any evidence showing that DCS has function on the therapeutic effects of levodopa in Parkinson's disease. It also did not affect the syndromes in Parkinson's disease, such as motor dysfunction, mental and psychotic deficits, emotional impairment, cognitive deficit and hallucination.

Moreover, the abstracts of U.S. Pat. No. 6,974,821 and U.S. Pat. No. 6,667,297 disclosed that DCS was used for treating schizophrenia and Parkinson's disease. However, they did not disclose that what syndrome DCS had improved and merely referred to autism. They did not refer to any other psychotic disease. U.S. Pat. No. 6,228,875 demonstrated that co-administration of DCS and neuroleptics had function on schizophrenia and Alzheimer's disease. However, it did not disclose what syndrome of these two diseases was affected. The U.S. Pat. No. 5,668,117 disclosed that combined administration of DCS and carbonyl trapping primary amine agents was used for treatmenting psychotic disorders. However, it did not disclose what syndrome was cured. In U.S. Pat. No. 5,486,763 and U.S. Pat. No. 5,087,633 claimed that DCS improved learning and memory deficits. U.S. Pat. No. 5,468,763 and U.S. Pat. No. 5,061,721 disclosed that the combination of DCS and specific ratio of D-alanine could improve learning, memory, and cognitive function. U.S. Pat. No. 5,187,171 disclosed DCS was used for the treatment of PCP-induced schizophrenia.

The inventor and the college recently were engaged in the related studies illustrated above, for example, involvement of NMDA receptors in both MPTP-induced neuroinflammation and deficits in episodic-like memory in Wistar rats. (Behav Brain Res, Online publish: 2009 Nov. 9.)

### BRIEF DESCRIPTION OF THE DRAWINGS

FIG. 1A illustrates the spatial configuration of objects in open field (: old object; [symbol] : recent object). FIG. 1B illustrates the time that rats spend on exploring the objects in example 1.

FIG. 2A illustrates the immunostaining diagrams of rat striatum in example 2. FIGS. 2A to 2D show the immunostaining stained with mouse monoclonal antibodies against rat tyrosine hydroxylase (TH). The immunostaining was observed by 50× microscope and the scale is 200 μl. FIG. 2B is the immunostaining of rat striatum in experimental group 0 (E0) in example 2. FIG. 2C is the immunostaining of rat striatum in experimental group 1 (E1) in example 2. FIG. 2D is the immunestaining of rat striatum in experimental group 2 (E2) in example 2. FIG. 2E is the relative optical density of immunostaining in rat striatum in example 2.

FIG. 3 illustrates the immunostaining of rat brain SNc in example 2. FIGS. 3A to 3D show the immunostaining stained with mouse monoclonal antibodies against rat tyrosine hydroxylase (TH). The immunostaining was observed by 50× microscope and the scale is 200 μl. FIG. 3B is the immunostaining of rat SNc in experimental group 0 (E0) in example 2. FIG. 3C is the immunostaining of rat SNc in experimental group 1 (E1) in example 2. FIG. 3D is the immunostaining of rat SNc in experimental group 2 (E2) in example 2. FIG. 3E is the density of DAergic neurons in rat SNc in example 2.

FIG. 4 illustrates the immunostaining of rat brain striatum in example 2. FIGS. 4A to 4D show the immunostaining stained with mouse monoclonal antibodies against rat MHC class II (OX-6). The immunostaining was observed by 50× microscope and the scale is 200 μl. The small square shows the staining observed by 200× microscope. FIG. 4B is the immunostaining of rat striatum in experimental group 0 (E0) in example 2. FIG. 4C is the immunostaining of rat striatum in experimental group 1 (E1) in example 2. FIG. 4D is the immunostaining of rat striatum in experimental group 2 (E2) in example 2. FIG. 4E is the density of activated microglia in immunostaining of rat striatum in example 2.

FIG. 5 illustrates the immunostaining of rat brain SNc in example 2. FIGS. 5A to 5D show the immunostaining stained with rat MHC class II antibody (OX-6). The immunostaining was observed by 50× microscope and the scale is 200 μl. The small square shows the staining observed by 200× microscope. FIG. 5B is the immunostaining of rat SNc in experimental group 0 (E0) in example 2. FIG. 5C is the immunostaining of rat SNc in experimental group 1 (E1) in example 2. FIG. 5D is the immunostaining of rat SNc in experimental group 2 (E2) in example 2. FIG. 5E is the density of activated microglia in immunostaining of rat SNc in example 2.

FIG. 6 illustrates the immunostaining of rat brain hippocampus in example 2. FIGS. 6A to 6D show the immunostaining stained with rat MHC class II antibody (OX-6). The immunostaining was observed by 50× microscope and the scale is 200 μl. The small square shows the staining observed by 200× microscope. FIG. 6B is the immunostaining of rat hippocampus in experimental group 0 (E0) in example 2. FIG. 6C is the immunostaining of rat hippocampus in experimental group 1 (E1) in example 2. FIG. 6D is the immunostaining of rat hippocampus in experimental group 2 (E2) in example 2. FIG. 6E is the density of activated microglia in immunostaining of rat hippocampus in example 2.

FIG. 7 illustrates the immunostaining of rat brain hippocampus in example 2. FIGS. 7A to 7D show the immunostaining of the pyramidal cells in the hippocampal CA1 area stained by Nissl stain. The immunostaining was observed by 200× microscope and the scale is 200 μl. FIG. 7B is the immunostaining of rat hippocampus in experimental group 0 (E0) in example 2. FIG. 7C is the immunostaining of rat hippocampus in experimental group 1 (E1) in example 2. FIG. 7D is the immunostaining of rat hippocampus in experimental group 2 (E2) in example 2. FIG. 7E is the percentage of neuronal area in the hippocampal CA1 area in example 2.

### SUMMARY OF THE INVENTION

The present invention provides a method of treating a neurodegenerative disorder in a patient, comprising administration to the patient a therapeutically effective amount of D-cycloserine or its derivatives.

### DETAILED DESCRIPTION OF THE INVENTION

Taken the above disclosure, they did not provide the use of D-cycloserine (DCS) or its derivatives for treating neuroinflammation, neurodegeneration, and cognitive deficits caused by DAergic degeneration.

Since the changes of neuronal function and cognitive deficits in MPTP-treated rats were similar to that seen in patients with Parkinson's disease, MPTP-treated rats were used to evaluate the function of D-cycloserine and its derivatives on Parkinson's disease and dementia in this disease. D-cycloserine improves neuronal and cognitive deficits caused by MPTP lesion. Besides, pathophysiological changes caused by MPTP are similar to that seen in other neurodegenerative disorders. They both show neuroinflammation and neuronal cell death and also cognitive deficits. Therefore, MPTP-lesioned rats are used as a model of neurodegenerative disorder. MPTP was used in the present invention for inducing neurodegeneration and then D-cycloserine was administered. The parameter of neuroinflammation was measured and used as an index of the effects of D-cycloserine and its derivatives on neurodegeneration. The present invention provides a use of D-cycloserine in treating neurodegenerative disorder.

The present invention provides a method of treating a neurodegenerative disorder in a patient, comprising an administration of therapeutically effective amount of D-cycloserine or its derivatives to the patient.

As refer to in the present invention, the term “neurodegenerative disorder” refer to neuroinflammation-related disorder, including, but are not limited to: Parkinson's disease, Alzheimer's disease, schizophrenia, apoplesia, multiple sclerosis, Lewy body dementia, frontal dementia and spinocerebellar ataxia.

As refer to in the present invention, the term “D-cycloserine derivative” includes the protective forms of ion, salt and solvate, for example, salt of D-cycloserine, ester of D-cycloserine, alkylated D-cycloserine, D-alanine and D-serine, but is not limited to the example herein. The preparation process of these compounds is known to the skilled in the art.

The D-cycloserine and its derivative in the present invention can manufacture into various forms including tablet, pellet, and coated tab (dragee). The D-cycloserine and its derivative can also be filled into proper container, such as capsule, or suspension and packed into bottle.

In addition, the D-cycloserine or its derivative in the present invention is administered alone or administered together with other therapeutic drug. The D-cycloserine or its derivative is administered by oral or non-oral, e.g. intravenous injection, trans-mucosal, sublingual administration, peritoneal administration, intrathecal administration and intramuscular injection, depending on the therapeutic requirement.

The present invention provides a method of treating neurodegenerative disorders in a patient, comprising administering an effective amount of D-cycloserine or its derivative, which can inhibit neuroinflammation and neuronal cell death, and improve cognitive deficit and psychotic syndrome. In the preferred embodiment, the effective amount of D-cycloserine in the present invention is 5 to 10 mg/kg per day in rats. In another preferred embodiment, the effective amount of D-cycloserine in the present invention is about 56 to 112 mg/kg per day in human (body weight of 70 kg), according to the conversion between rat and human.

The “inhibition of neuroinflammation” in the present invention refers to inhibiting the activation of microglia.

The cognitive function in the present invention refers to the similar function as listed below: function of learning, memory, contextual memory, object recognition, visuospatial recognition, and executive function.

The psychotic syndrome in the present invention refers to sensory hallucination or similar syndrome, for example, visual hallucination and auditory hallucination.

### EXAMPLE

The examples below are non-limiting and are merely representative of various aspects and features of the present invention.

### Example 1

Thirty-nine male Wistar rats (306.2±0.9 g; BioLASCO Taiwan Co., Ltd.) were used as experimental animals in the present invention. All experimental procedures were performed according to the NIH Guide for the Care and Use of Laboratory Animals and were approved by the Animal Care Committee of the Chung Shan Medical University (IACUC approval No.: 434). All animals underwent stereotaxic surgery and MPTP were injected into the substantia nigra pars compacta (SNc) in the animals to establish Parkinson's disease animal model. One day after the surgery, the rats received daily dose of intraperitoneal (i.p.) injections of D-cycloserine (DCS) (0, 5, or 10 mg/kg/day) or saline at 18:00 for 13 days. The animals were divided respectively into groups of experimental group 0 (E0), receiving MPTP+saline, experimental group 1 (E1), receiving MPTP+DCS 5 mg/kg/day, experimental group 2 (E2), receiving MPTP+DCS 10 mg/kg/day and Sham-operated group/control group (CR), receiving saline (1 ml/kg/day, i.p.) (also called control group or Sham+saline group). The descriptions of “control group, experimental group 0, experimental group 1 and experimental group 2” were used below.

For the brain surgery, all animals were intraperitoneal injected with Zoletil (2 mg/kg; Virbac, Carros, Franc) and received stereotaxic surgery after anesthetization. MPTP-HCl (1 μmol in 2 μl of saline; Sigma, Mo., USA) (Da Cunha et al., Behav Brain Res, 2001, 124:9-18; Gevaerd et al., Brain Res Bull, 2001, 55: 101-6) or saline (2 μl) were microinjected into the substantia nigra pars compacta (SNc).

### Episodic-Like Memory Test

The episodic-like memory test was performed in an open field (60×60×40 cm, without roof allowing rats to aware external distal cues), 13 days after the MPTP lesion. The floor of the open field was divided into 9 areas of equal size. A video camera was mounted 160 cm above the center of the open field to record and analysis the behavior of rats. Two different sets of objects made of metal or plastic were used in the experiment. The objects weighed about 950 g and therefore, they were unmovable by the rats. The two objects differed in the diameter of base, height, color, shape, and surface texture (the metal object: column in shape, 6.5 cm in diameter, 12 cm high, silver-colored, smooth surface; the plastic object: cuboid in shape, a 7.5×5 cm cross section, 15 cm high, pellucid-colored, rough surface). One day before the test, rats were placed individually into the open field for twice and allowed to explore it (once for 5 min), with a 4 h interval, for adapting to the testing environment.

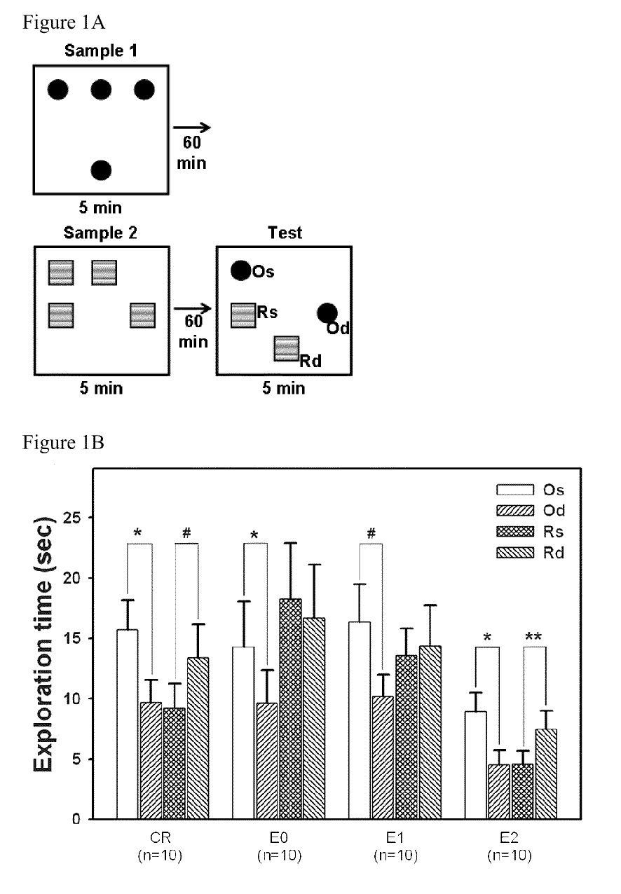

The procedure of episodic-like memory test was consistent with previous reported research (Li and Chao., Neurobiol Learn Mem 2008; 89:192-8). The method comprised two sample trials and one testing trial. In the first sample trial, the rats were placed in the open field with four identical objects (FIG. 1A, upper left), and allowed to explore them for 5 min. After an interval of 60 min, the rats were put into the same open field again and received the second sample trial for 5 min, wherein the objects were replaced with another objects and the spatial configuration was changed (FIG. 1A, lower left). After an additional interval of 60 min, the rats received a testing trial, wherein two identical objects from sample trial 1 (the “old” objects; “O”) and two identical objects from sample trial 2 (the “recent” objects; “R”) were presented. In the testing trial, animals encountered a mixed set consisting of two “O” and two “R” objects. One object from each of the two sets of objects was placed in the “same” location as in the sample trials (Os and Rs). The other two objects were “displaced” to new locations (Od and Rd) (FIG. 1A, lower right). Time spent on exploring the objects was recorded.

One-way ANOVA showed that total object exploration time in both sample trial 1 and sample trial 2 in the episodic-like memory test was not significantly different between the groups (both P-values >0.05, data not shown). In the test trial, total exploration time of Control/Sham+saline (CR) (48.0±8.2 sec), MPTP+saline (E0) (58.8±14.7 sec), MPTP+DCS 5 (E1) (54.4±5.9 sec), and MPTP+DCS 10 (E2) (25.4±4.3 sec) groups showed no significant differences between groups (F(3, 35)=2.397, P=0.085), but exhibited a tendency of DCS treatment therein. The 2 (object recency)×2 (location) ANOVA with two within-subject factors was carried out for the four groups separately. There were “object recency by location” interactions in the control group (CR) (Sham+saline, F(1, 9)=13.747, P=0.005), experiment group 1 (E1) (MPTP+DCS 5, F(1, 9)=5.795, P=0.039), and experiment group 2 (E2) (MPTP+DCS 10, F(1, 8)=22.252, P=0.002), respectively. However, no such interaction was found in the control group (CR) (F(1, 9)=1.516, P=0.249). FIG. 1B illustrated the object exploration time in each group. A significant main effect of object recency was found in experimental group 0 (E0) (MPTP+saline rats, F(1, 9)=6.736, P=0.029) but not in other groups, shown in FIG. 1B. Furthermore, no main effect of location was found in any group. A paired t-test revealed that rats in control group (CR) (d.f.=9, t=3.051, P<0.05), experimental group 0 (E0) V.=9, t=2.897, P<0.05), and experimental group 2 (E2) (d.f.=8, t=3.249, P<0.05) spent a longer time exploring the old object placed in the same location (Os) than exploring the old object placed in a different location (Od). The time spent on Os and Od in experimental group 1 (E1) rats had the trend of significance (d.f.=9, t=2.101, slightly less than the critical value 2.69) (FIG. 1B).

For the recent objects, a paired t-test revealed that rats in both the control group (CR) (Sham+saline group, d.f.=9, t=−2.614, slightly less than the critical value 2.69) and the experimental group 2 (E2) (MPTP+DCS 10 group, d.f.=8, t=−4.028, P<0.001) spent a longer time exploring the displaced object (Rd) than exploring the object located in the same place (Rs). However, no differences between exploring Rs and exploring Rd were found in either the experimental group 0 (E0) or the experimental group 1 (E1).

The results above showed that the rats in the control group were able to perform the episodic-like memory. They could distinguish the difference between recent object and old object and their locations. However, MPTP lesion caused impairments in the behavior described above. The function of episodic-like memory was recovered by the treatment of DCS, at the dosage of 10 mg/kg/day.

### Example 2

### Histological Assay and Image Analysis

One day after the test of episodic-like memory, rats were sacrificed under deep anesthesia by CO2. For histological assessment, 5-6 randomly selected rats per group were perfused cardiacly with 4% paraformaldehyde in phosphate-buffered saline (PBS), and the brains were removed rapidly and post-fixed in 20% sucrose solution with 4% paraformaldehyde at 4° C. The frozen coronal brain sections were cut into 30 μm and further immunostained with mouse monoclonal antibodies against rat tyrosine hydroxylase (TH) (1:2000; Zymade, USA) or rat MHC class II (OX-6; 1:200; BD Biosciences Pharmingen, CA, USA) at 4° C. overnight, identical to the method used in our previous report (Wang, Wu, Liou, et. al, Behav Neurosci, 2009). In sections containing hippocampus, neurons in the hippocampus was identified by Nissl staining method.

A microscope (ZEISS AXioskop2, Germany) disposed with a CCD (Optronics, USA) and the Image Pro Plus Software 6.0 (Media Cybernetics, CA, USA) was used for observation. The three square areas (measuring 36,477, 18,769, and 2,354 μm²) were applied respectively to determine the relative optical density of TH immunoreactivity in the striatum and the neuronal density in the SNc and hippocampal CA1 area. The images of TH staining were converted to the gray scale for measuring the density of DAergic projections. The gray level of a given area of interest was measured, and the background staining measured in non-immunoreactive corpus callosum was subtracted. Thus, the relative optical density of the TH stained tissue was obtained. For further measuring the density of DAergic neurons in the SNc, the images were captured, and an area of interest was overlaid in this region. The somas of TH immunoreactive neurons located in this area were counted. The density of activated microglia was measured, according to the methods described in the literature (Sugama S, et al., Brain Res, 2003, 964: 288-94). The brain sections were sampled at equidistant positions. Then, the numbers of activated microglial cells in the SNc, striatum, and hippocampus were counted in the areas of interest (measuring 18,769, 36,477, and 18,769 μm², respectively). Because the neurons were tightly packed, it was difficult to directly count the number of pyramidal neurons in the CA1 area from a 30 μm-thick brain section. Thus, the neuronal density was represented by a semi-quantitative method, calculating the percentage of an area occupied by Nissl-stained neurons in an area of interest in the CA1 area.

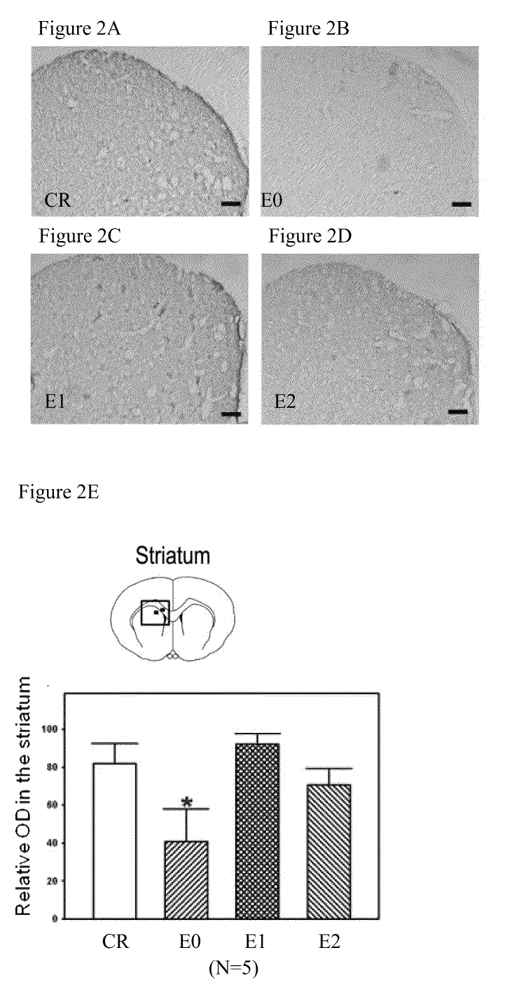

FIG. 2 illustrates the effect of DCS on the striatum of rat brain lesioned by MPTP. The TH staining of DAergic neurons is shown in FIGS. 2A-2D. The relative optical density of stained tissue is illustrated in FIG. 2E. * P<0.05, compared with the control group, CR.

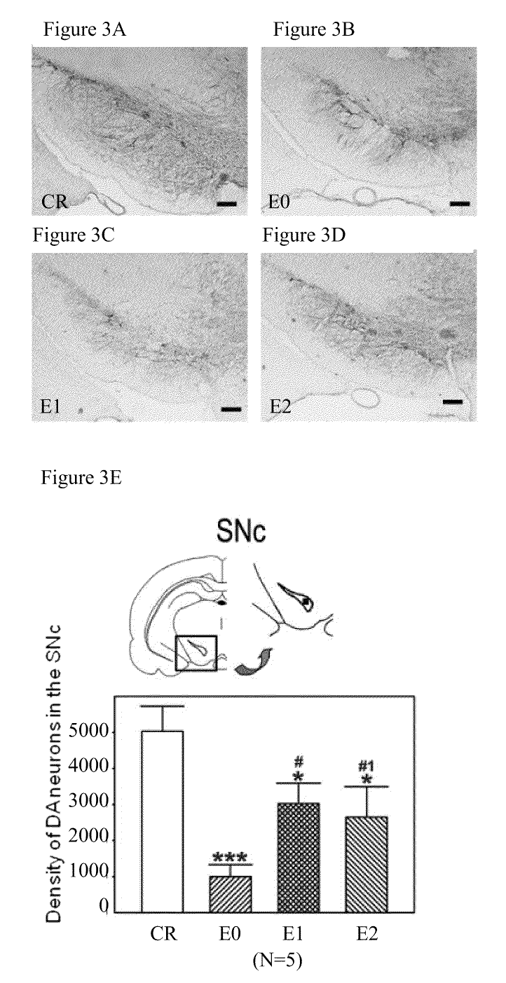

FIG. 3 illustrates the effect of DCS on the SNc of rat brain lesioned by MPTP. TH staining of DAergic neurons is shown in FIGS. 3A-3D. The density of DAergic neurons is illustrated in FIG. 3E. * P<0.05, *** P<0.001, compared with the control group, CR. # P<0.05, #1 P=0.081, compared with the experimental group 0, E0.

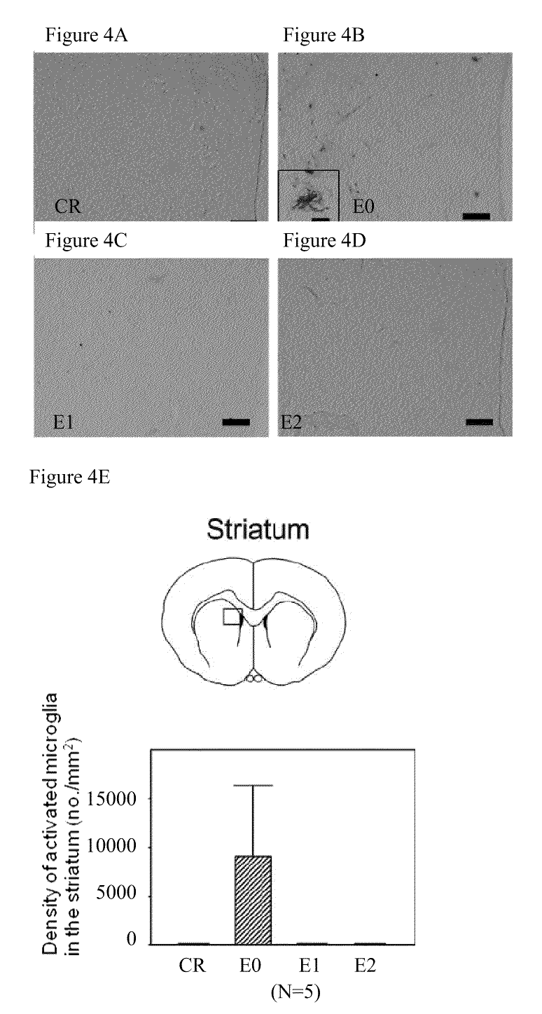

FIG. 4 illustrates the effect of DCS on the activation of microglia in the striatum induced by MPTP lesion. The activation of microglia was labeled by OX-6, shown in FIG. 4A-4D. The density of activated microglia is illustrated in FIG. 4E.

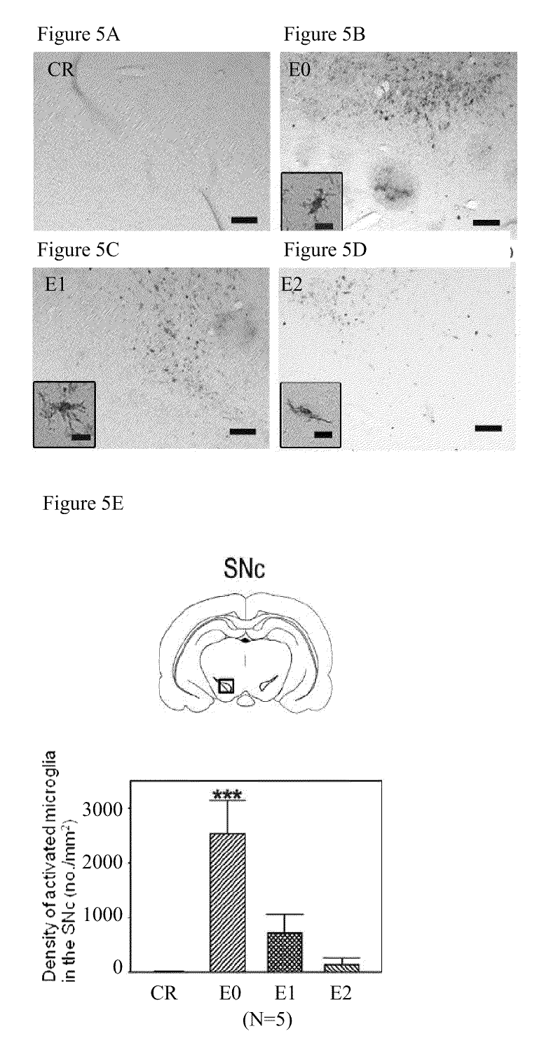

FIG. 5 illustrates the effect of DCS on the activation of microglia in the SNc induced by MPTP lesion. The activation of microglia was labeled by OX-6, shown in FIG. 5A-5D. The density of activated microglia is illustrated in FIG. 5E. *** P<0.001, compared with the control group, CR.

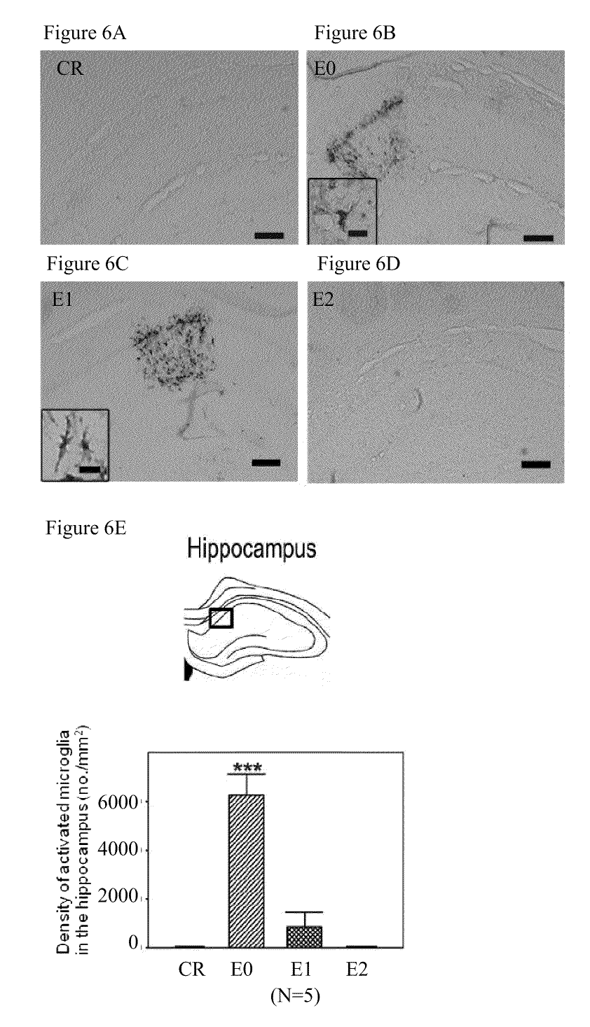

FIG. 6 illustrates the effect of DCS on the activation of microglia in the hippocampus induced by MPTP lesion. The activation of microglia was labeled by OX-6, shown in FIG. 6A-6D. The density of activated microglia is illustrated in FIG. 6E. *** P<0.001, compared with the control group, CR.

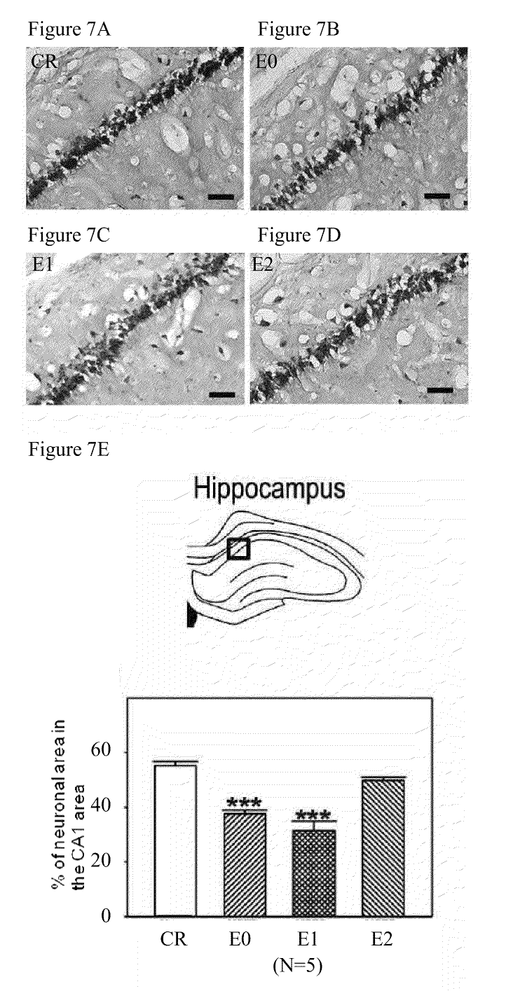

FIG. 7 illustrates the effect of DCS on cell loss in the hippocampal CA1 area induced by MPTP lesion. The Nissl staining of the pyramidal cell layer in the hippocampal CA1 area is shown in FIGS. 7A-7D. The percentage of an area occupied by Nissl-stained neurons in an area was calculated by semi-quantitative method. *** P<0.001, compared with control group, CR.

TH immunoreactivity in the SNc and striatum were shown. The resolution of the TH staining was sufficient for counting the cell number in a specific area under light microscopy. Semi-quantitative analysis confirmed that MPTP lesions induced a reduction in relative optical density of TH immunoreactivity in the striatum (P=0.020) (FIGS. 2B and 2E) and a reduction in the density of DAergic neurons in the SNc (P<0.001) (FIGS. 3B and 3E). DCS treatment at the dosage of 5 and 10 mg/kg/day blocked the MPTP-induced DAergic degeneration described above (FIGS. 2C, 2D, 2E, 3C, 3D, and 3E). FIG. 5 and FIG. 6 illustrate that MPTP lesion increased microglial activation in the SNc and hippocampus. Semi-quantitative analysis showed that the density of activated microglia in the SNc (FIGS. 5B and 5E) and hippocampus (FIGS. 6B and 6E) in the experimental group 0 (MPTP+saline group, E0) was higher than that in the control group, CR (both P-values <0.001). The treatment of DCS at the dosage of 5 and 10 mg/kg/day, suppressed MPTP-induced microglial activation in the SNc (FIGS. 5C, 5D, and 5E) and hippocampus (FIGS. 6C, 6D, and 6E). As shown in FIG. 4, the microglial activation in the striatum was induced by the lesion of MPTP. However, it showed no significant difference between the groups. As shown in FIG. 7, MPTP lesion decreased the neuronal density in the pyramidal cell layer in the hippocampal CA1 area, compared to the control group, CR (P<0.001). DCS treatment at the dosage of 10 mg/kg/day blocked the above MPTP-induced neurodegeneration.

The above results indicated that MPTP lesion damaged the DAergic system, induced neuroinflammation and microglial activation, and caused neurodegeneration in the hippocampus. The treatment of DCS inhibited the above DAergic neurodegeneration and cell loss in the hippocampus, possibly through inhibition of the neuroinflammation and microglial activation.

Given the above, DCS inhibited MPTP-induced DAergic neurodegeneration, microglial activation, and cell loss in the hippocampus. In addition, DCS can also inhibit MPTP-induced deficits in episodic-like memory and impairment of recognition.

While the invention has been described and exemplified in sufficient detail for those skilled in this art to make and use it, various alternatives, modifications, and improvements should be apparent without departing from the spirit and scope of the invention.

One skilled in the art readily appreciates that the present invention is well adapted to carry out the objects and obtain the ends and advantages mentioned, as well as those inherent therein. The animals and methods for producing them are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

What is claimed is:

1. A method of treating a neurodegenerative disorder in a patient, comprising administration to the patient a therapeutically effective amount of D-cycloserine or its derivatives.

2. The method of claim 1, wherein the D-cycloserine derivatives are selected from the group consisting of: salt of D-cycloserine, ester of D-cycloserine, alkylated, D-alanine or D-serine.

3. The method of claim 1, wherein the D-cycloserine or its derivatives is administered alone or administered with other therapeutic drugs.

4. The method of claim 1, wherein the administration of the D-cycloserine or its derivatives is oral administration, intravenous injection, trans-mucosal administration, sublingual administration, peritoneal administration, intrathecal administration, or intramuscular injection.

5. The method of claim 1, wherein the administration of D-cycloserine is in a amount of 56 to 112 mg/day.

6. The method of claim 1, wherein the neurodegenerative disorder is Parkinson's disease, Alzheimer's disease, schizophrenia, apoplesia, multiple sclerosis, Lewy body dementia, frontal dementia or spinocerebellar ataxia.

7. The method of claim 1, wherein the treating of the neurodegenerative disorder is through an inhibition of neuroinflammation.

8. The method of claim 7, wherein the inhibition of neuroinflammation is inhibiting the activation of microglia.

9. The method of claim 1, which inhibits central neurodegeneration or neuronal cell death in neurodegenerative disorder.

10. The method of claim 1, which improves cognitive function and psychotic syndrome.

11. The method of claim 10, wherein the cognitive function is of learning, memory, contextual memory, object recognition, visuospatial recognition, or executive function.

12. The method of claim 10, wherein the psychotic syndrome is hallucination.

13. The method of claim 10, wherein the hallucination is visual hallucination, auditory hallucination or other sensory hallucination.
